(19) United States
(12) Patent Application Publication
(10) Pub. No.: US 2006/0276690 A1
(43) Pub. Date: Dec. 7, 2006

(54) Visualization system

(76) Inventors: Jesse Len Farris III, Andover, MA (US); David I. Freed, Westborough, MA (US); John Gauvin, Southbridge, MA (US); James E. McDonald, Monson, MA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/432,287

(22) Filed: May 11, 2006

Related U.S. Application Data
(60) Provisional application No. 60/680,143, filed on May 11, 2005.
(60) Provisional application No. 60/680,845, filed on May 12, 2005.

(51) Int. Cl. A61B 1/06

(52) U.S. Cl. 600/162

(57) ABSTRACT

An interface connection between a fiberscope and an ocular includes a post and a bore that form a male and female connector between the fiberscope and the ocular. The post and bore also include a helical slot and a pin that fits to secure the post within the bore. In another aspect of the invention, a holder including an elastic C-shaped connector is releasably secured to an ocular.

### CROSS-REFERENCE TO RELATED APPLICATIONS

This application claims the benefit of U.S. Provisional Application No. 60/680,143, filed May 11, 2005, and No. 60/680,845, filed on May 12, 2005, which are herein incorporated by reference.

### FIELD OF THE INVENTION

Embodiments of the present invention generally relate to visualization systems for use in medical or industrial applications, such as fiberscopes or borescopes.

### BACKGROUND

Visualization devices, such as fiberscopes and borescopes, are well known in the exploration and treatment of internal areas of the human anatomy or inspection of internal spaces of industrial equipment. Fiberscopes and borescopes are elongated optical devices capable of transmitting visual images to a remote observer. The devices are used to transmit images of objects near the distal end of the device to an observer at the near or proximal end of the device. This image transmitting capability permits an observer to perform a visual inspection of remote objects within the field of view of the far end of the device. Fiberscopes and borescopes are typically used to permit the inspection of a large variety of objects located in remote, inaccessible or hazardous areas, such as internal body cavities, vasculature, or lumens of patients and industrial equipment, such as turbine engines and nuclear reactors.

Most modem fiberscopes and borescopes utilize flexible fiber-optic cables within a shaft. The fiber-optic cables contain a bundle of parallel transparent fibers that transmit an image from one end of the shaft to the opposite end of the shaft. The fiber-optic fiberscopes and borescopes generally include a protective sheath covering the entire length of the shaft. Most fiber-optic fiberscopes and borescopes also include a second fiber-optic bundle, which is illuminated by a bright light source at the observer's end. The illumination bundle transmits light to the remote end of the shaft, which then illuminates the field of view of the imaging fibers.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a perspective view of one exemplary embodiment of a visualization system constructed in accordance with the present invention;

FIG. 2 is a perspective view of one embodiment of a visualization probe, including a proximal connector in accordance with an embodiment of the present invention;

FIG. 3 is a more detailed view of a proximal connector for the visualization probe illustrated in FIG. 2;

FIG. 4 is a side view shown partially in cross-section of one embodiment of an ocular device constructed in accordance with aspects of the present invention;

FIG. 5 is a front perspective view of the ocular device shown in FIG. 4;

FIG. 6 is a partial cross-sectional view of one embodiment of a connection interface between a visualization probe and an ocular device constructed in accordance with an embodiment of the present invention;

FIG. 7 is a top perspective view of the ocular device shown in FIG. 4;

FIG. 8 is a bottom perspective view of the ocular device shown in FIG. 4;

FIG. 9 is a cross-sectional view of the ocular device shown in FIG. 4 at the location of a holder interface;

FIG. 10 is a rear perspective view of a holder constructed in accordance with an embodiment of the present invention;

FIG. 11 is a cross-sectional view of the holder of FIG. 10; and

FIG. 12 is a perspective view of the ocular device of FIG. 7 connected to the holder of FIG. 10.

### DETAILED DESCRIPTION

Embodiments of the present invention will now be described with reference to the drawings where like numerals correspond to like elements. Embodiments of the present invention are directed to systems of the type broadly applicable to numerous medical applications in which it is desirable to insert one or more steerable or non-steerable imaging devices into a body lumen or passageway. While the disclosed embodiments are described as suitable for use in medical procedures, aspects of the present invention have wide application, and therefore, may find uses in other fields, such as industrial applications, where the use of visualization systems is highly desirable. Accordingly, the following descriptions and illustrations herein should be considered illustrative in nature, and thus, not limiting the scope of the present invention.

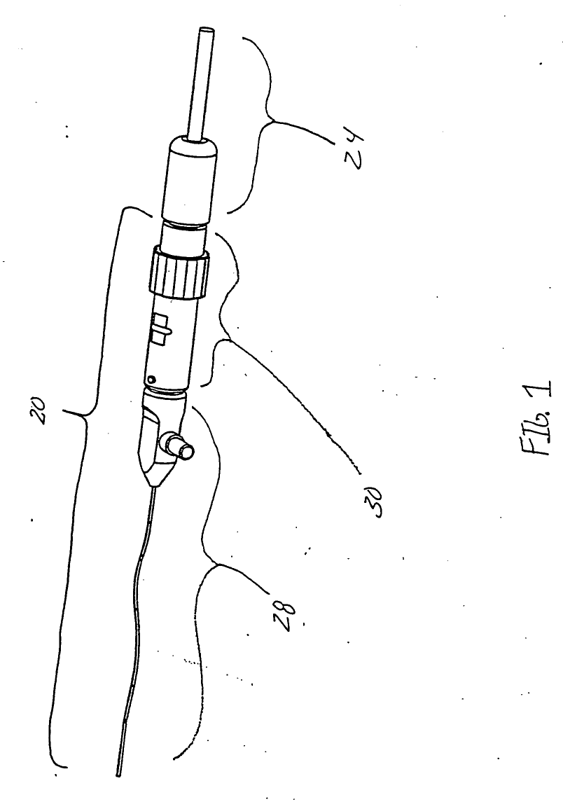

FIG. 1 is a perspective view of one illustrative embodiment of a visualization system 20 formed in accordance with aspects of the present invention. The visualization system 20 shown in FIG. 1 is adapted for connection to a video camera 24 or the like for displaying images on a video monitor or to an eyepiece for direct visualization. The visualization system 20 includes a visualization probe 28 selectively connected to an ocular device 30 via a proximal connector that will be described in more detail below. As such, the visualization probe 28 can be detached from the ocular device 30 after a procedure is complete for cleaning or sterilization and reuse. Alternatively, the visualization probe 28 can be discarded. Similarly, the ocular device 30 may be cleaned and reused.

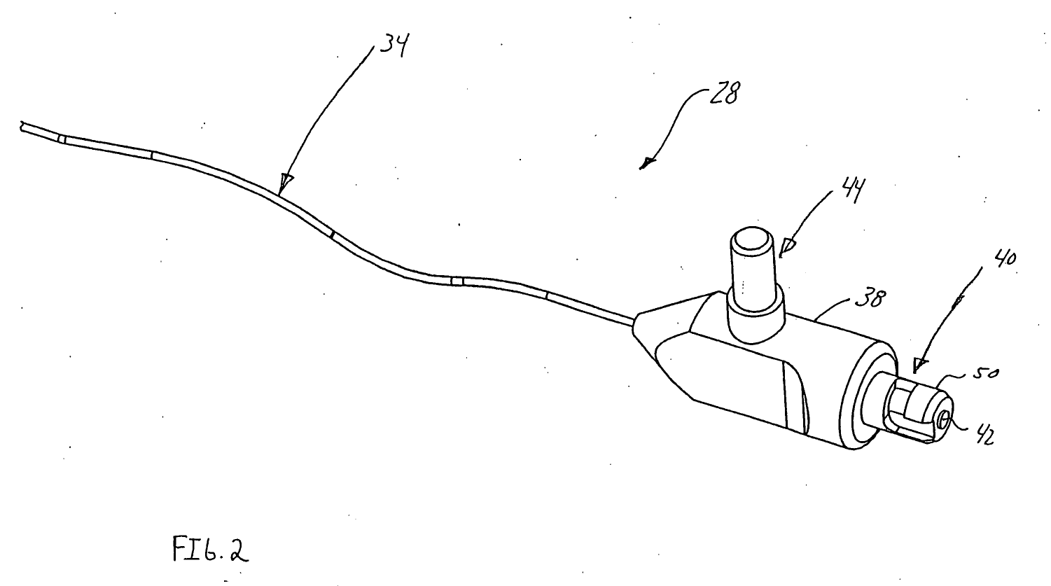

As best shown in FIG. 2, the visualization probe 28 comprises an elongate shaft 34 that is connected at its proximal end to a proximal connector 38. The shaft 34 includes, for example, one or more optical imaging fibers or bundles (not shown) that are encased by a cylindrical, elongated tubular sleeve. The outer diameter of the shaft 34 is preferably between 0.4 mm and 1.2 mm, although other sizes may be constructed depending on its intended application. The tubular sleeve of the shaft 34 may be constructed of any suitable material, such as polyimide or polyurethane, just to name a few. Additionally, a metallic hyptotube may be used.

In one embodiment, the shaft 34 includes one or more coherent imaging fibers or fiber bundles and one or more illumination fibers or fiber bundles (which may or may not be coherent). In some embodiments the illumination fibers surround the one or more imaging fibers. However, the illumination fibers may comprise any number of fibers and may be arranged in any configuration determined to be useful for illuminating a given procedure. The illumination fibers or fiber bundles may or may not be attached to the tubular sleeve via any suitable adhesive. The distal end of the shaft 34 may also include an image focusing lens and/or a window (not shown) that encloses the distal end to protect the fibers and to project the image from the field of view onto the distal end of the imaging fibers. In use, light transmitted by the illumination fibers illuminates the area or objects to be viewed, while the imaging fibers communicate the illuminated image to an image viewing device.

As was discussed above, the proximal end of the shaft 34 is functionally connected to a proximal connector 38. The proximal connector 38 preferably defines a bore (not shown) extending substantially therethrough, and in one embodiment, generally along its central, longitudinal axis. The bore may comprise internal threads or surface features adapted to securely engage the proximal end of the shaft 34, using adhesive, although other means for connecting the proximal end of the shaft 34 to the proximal connector 38 may be used. The proximal ends of the imaging fibers extend substantially entirely through the proximal connector 38, via the bore, and may extend slightly beyond the proximal end 40 of the proximal connector 38 as indicated at 42. The imaging fibers may be secured therein via suitable adhesive. In one embodiment, the proximal connector 38 further includes a light post 44 that is functionally connected to the proximal end of the illumination fibers. The light post 44 is configured to be releasably connected to a light cable (not shown) for supplying light from a light source to the illumination fibers.

The proximal connector 38 at its proximal end 40 terminates as a cylindrical protrusion or post 50, which in one embodiment is coaxially arranged with the central, longitudinal axis of the proximal connector 38. As will now be described in detail, the post 50 and features defined thereby form the male portion of the connector that selectively secures the proximal connector 38 to the ocular device 30 (See FIG. 1).

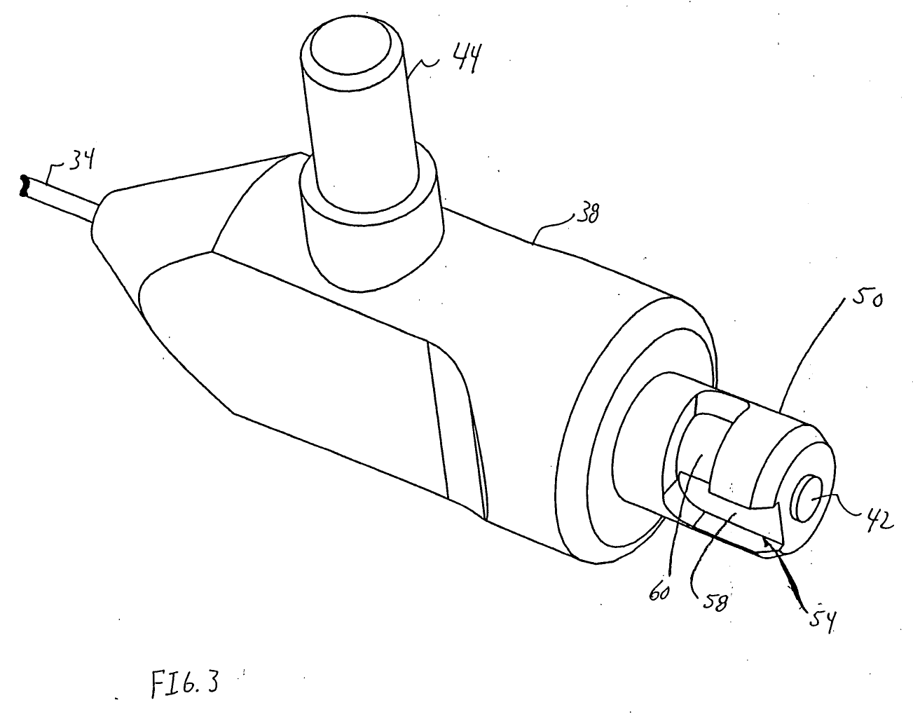

Turning now to FIG. 3, a somewhat L shaped slot 54 is formed in the post 50. The L-shaped slot 54 comprises a longitudinal section 58 and a transverse section 60. The longitudinal section 58 of the slot 54 begins at the end face of the post 50 and extends toward the distal end of the post 50 substantially parallel to the longitudinal axis of the post 50. The transverse section 60 of the slot 54 begins at the distal end of the longitudinal section 58 and extends around a portion of the post 50 (for example by approximately a ¼ turn) transversely with respect to the longitudinal axis of the post 50. In the embodiment shown, the transverse section 60 may be defined as a low-angle spiral or partially helical section. In one embodiment, the pitch angle of the spiral section is approximately 3° (three degrees) and has a helical direction from the proximal end to the distal end. In an alternative embodiment, the transverse section 60 of the slot may extend at a right angle to the longitudinal section 58. The C-shaped slot may have a constant depth or the depth may vary such that an increasing friction fit is formed between the slot and the pin or protrusion as the post is fitted into the bore. For example, the longitudinal section may have a constant depth while the transverse section may have a decreasing depth toward the end of the transverse section. In yet another embodiment, the slot may include a shallow depression that receives a spring loaded pin or protrusion to secure the post in the bore.

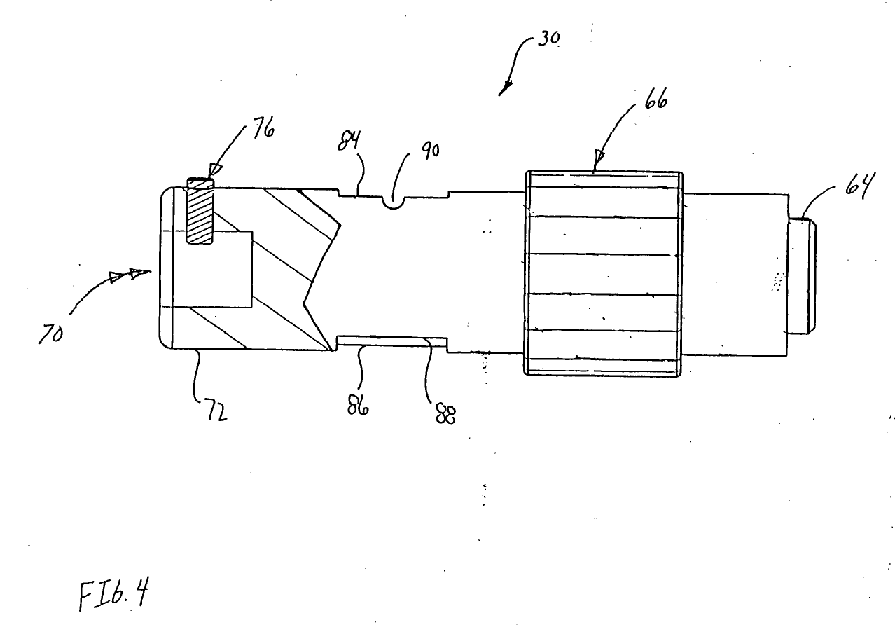

Returning to FIG. 1, the visualization system 20 further includes an ocular device 30 having one or more lenses (not shown) disposed in a conventional manner. In the embodiment shown in FIG. 4, the proximal end 64 of the ocular device 30 is configured to be selectively connected to a camera or imaging system such that users can save images and view them on a display. Alternatively, the ocular device 30 may be configured with an eyepiece for directly viewing images transmitted through the imaging fibers. It will be appreciated that the ocular device 30 may include other known components, such as an adjustment knob 66 for adjusting the relative positioning of one or more ocular lenses and, thus, adjusting the focus of the image transmitted through them.

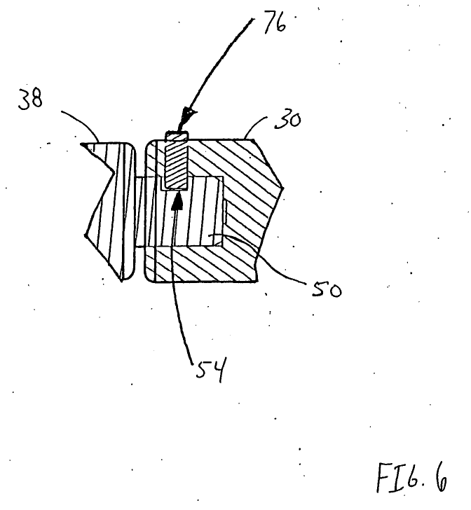

As is described above, the ocular device 30 is selectively secured to the visualization probe 28 (See FIG. 1) during use and may be separated after use to be cleaned while the visualization probe 28 may be either cleaned or sterilized for reuse or discarded. To that end, the ocular device 30 includes a cylindrical socket bore 70 disposed at its distal end 72. In one embodiment, cylindrical socket bore 70 is coaxial with the central longitudinal axis of the ocular device 30. The socket bore 70 is sized to slidably receive the post 50 of the proximal connector 38 in a supporting manner, as best shown in FIG. 6. As best shown in FIGS. 4 and 6, the ocular device 30 further includes a pin 76 or other protrusion positioned orthogonally to the longitudinal axis of the ocular device 30 and extending radially into the socket bore 70. The pin or protrusion may be fixed or movable, such as by spring loading it. The diameter of the pin 76 and the distance to which the pin 76 extends into the socket bore 70 are selected so as to be cooperatively received within the slot 54 in a slideable manner. As such, the socket bore 70 and pin 76 arrangement form the female portion of a connector that joins the ocular device 30 to the proximal connector 38 on the visualization probe 28.

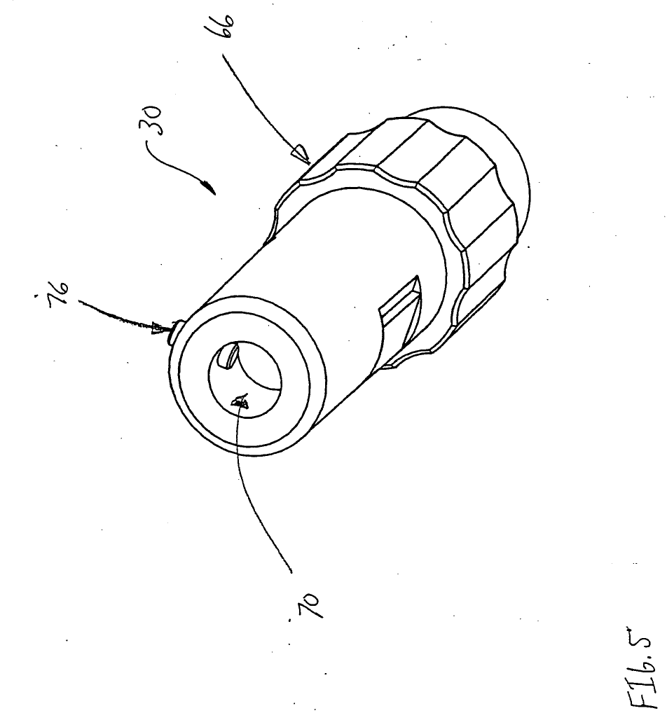

One technique for connecting the visualization probe 28 and the ocular lens device 30 will now be described with reference to FIGS. 1-6. To connect the proximal connector 38 to the ocular device 30, the proximal connector 38 is held in one hand while the ocular device 30 is held in the other hand or secured to an arm or other stationary device. Next, the proximal end of the proximal connector 38 is positioned in close proximity to the distal end 72 of the ocular device 30. The post 50 is then oriented with the socket bore 70 such that slot section 54 is aligned with the pin 76. Once alignment is achieved, the post 50 of the proximal connector 38 may be inserted into the socket bore 70. When the post 50 is fully inserted into the socket bore 70 such that the pin 76 engages the end of the longitudinal section 58 of the slot 54, the proximal connector 38 may then be rotated so that the pin 76 enters the transverse section 60 of the slot 54. Due to the helical configuration of the transverse section 60, the pin 76 engages the side of the transverse slot section 60 and draws the proximal connector 38 further into the ocular device 30, thereby achieving a locked position with a friction fit as the proximal connector 38 is rotated with respect to the ocular device 30. To decouple the components, the proximal connector 38 is rotated in the opposite direction, which then allows the post 50 to be removed from the socket bore 70.

In yet another embodiment of the invention, the slot 54 is included in the bore and the post 50 includes an outwardly extending pin 76 that fits within the slot.

In one suitable use of the visualization system 20, the ocular device 30 may be advantageously supported in a non-rotational position via a support arm, which may be mounted on a video cart or other suitable structure. Alternatively, the support arm could be configured as a hands-free type unit that the user could wear during a procedure to allow him/her to view images through the lens device without holding it. To that end, the ocular device 30 may further include interface features disposed along a portion of its length for detachably connecting to the support arm.

As best shown in FIGS. 4, 8 and 9, one embodiment of the interface features on the ocular device comprise one or more of two opposite facing flat surfaces 84 and 86, a stepped portion 88 which may be positioned adjacent the flat surface 86, and a semi-circular groove 90. In the embodiment shown, the groove 90 bisects the flat surface 84. In one embodiment, the groove 90 is transversely disposed with respect to the longitudinal axis of the ocular device 30 and functions as a guide when being coupled to a holder, as will be described in more detail below.

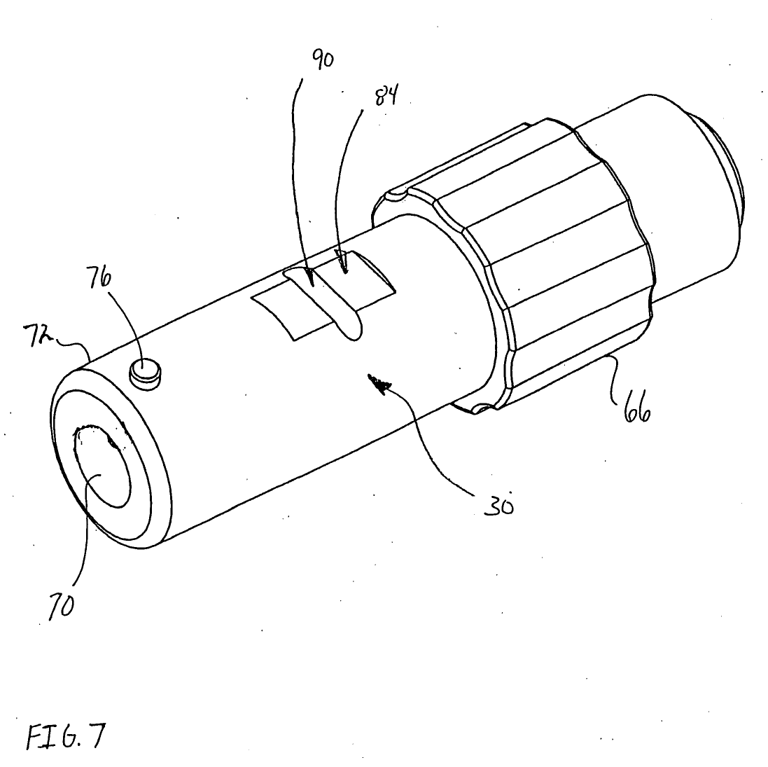

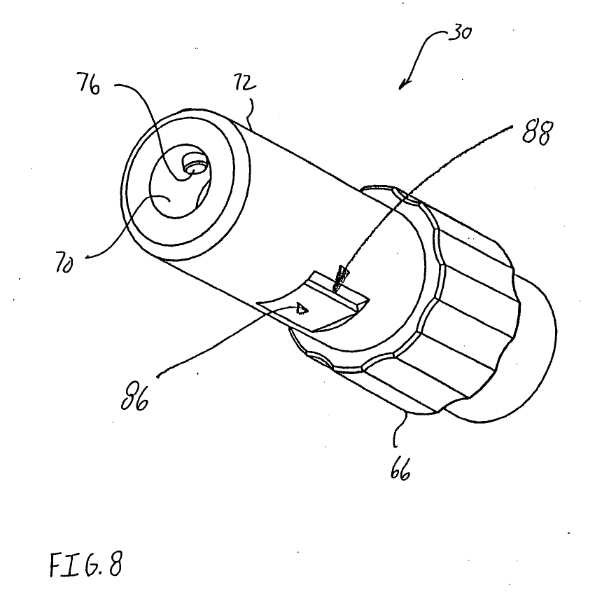

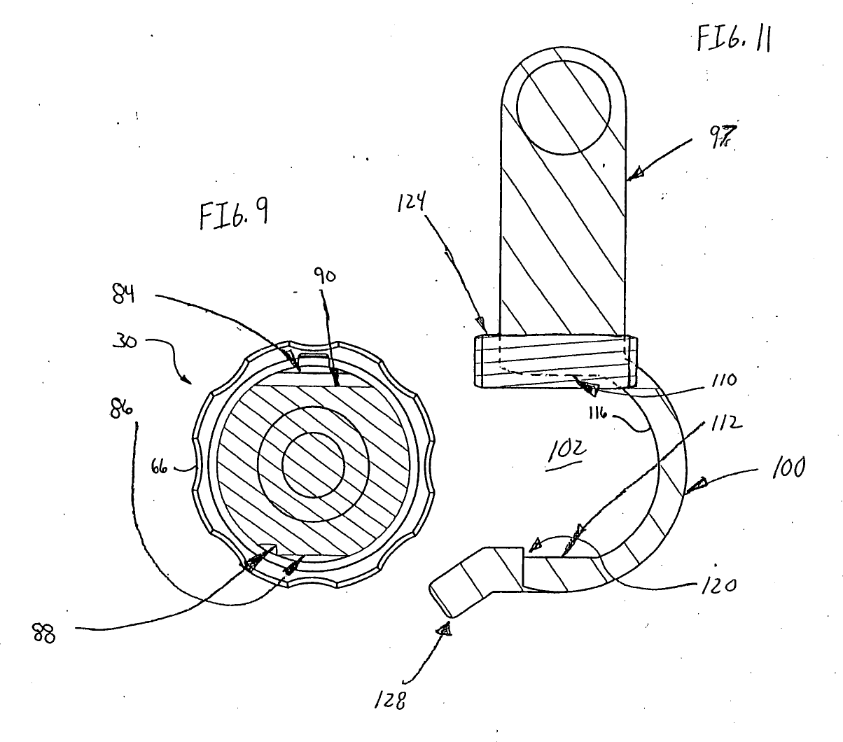

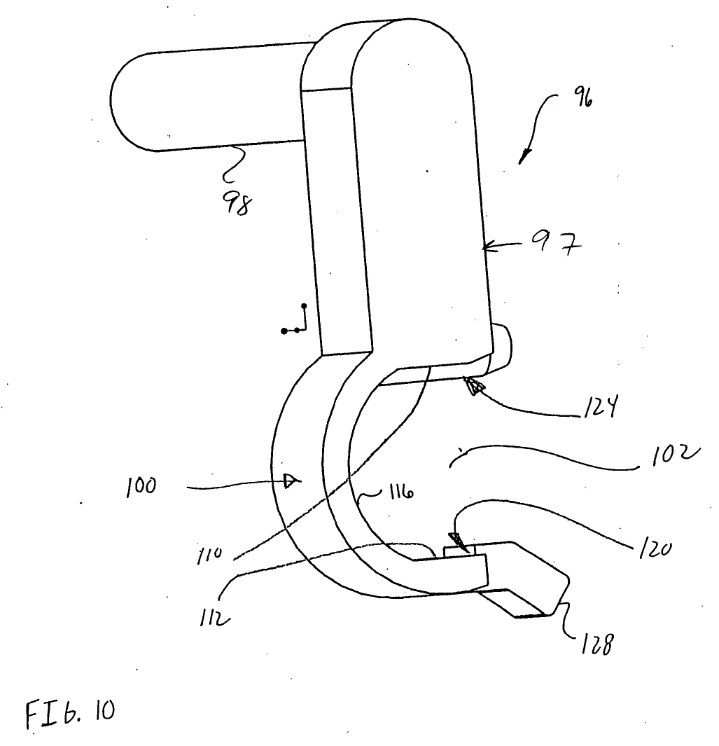

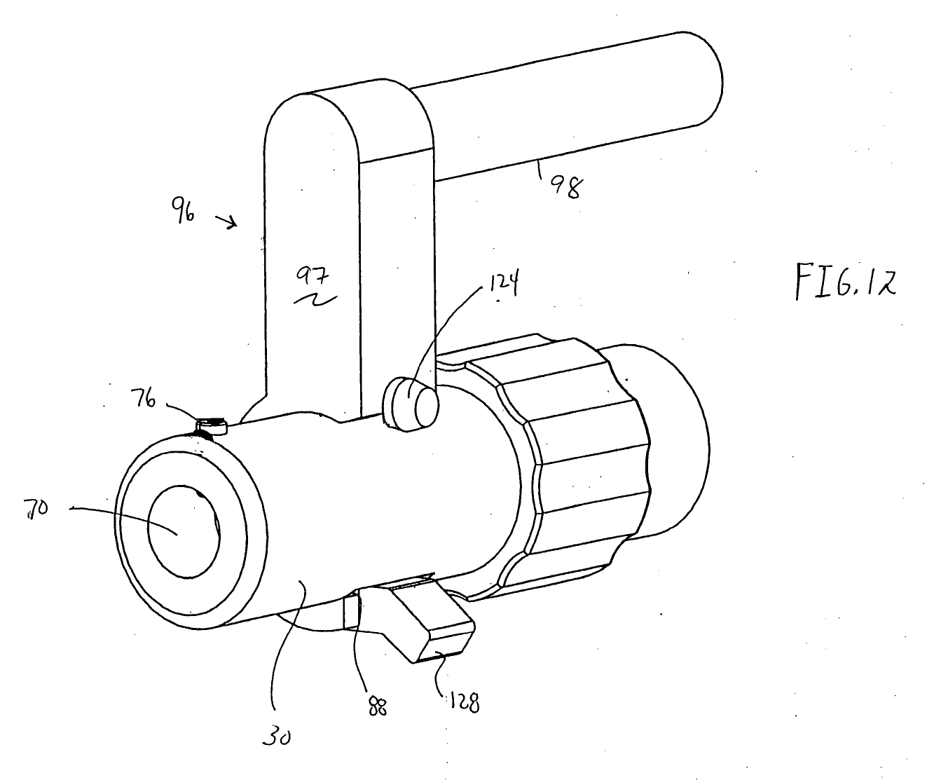

Referring now to FIGS. 10 and 11, a holder 96 includes a shank section 97 and a C-shaped connector at the end of the shank. In one embodiment, the other end of the shank 97 includes an outwardly extending arm 98 that can be fitted into a support arm. In other embodiments, the shank may include a hole or other structure that allows the shank to be secured to a desired object. The C-shaped connector 100 that defines an opening 102 is cooperatively sized and configured for receiving the side of the ocular device 30 in a locking manner, with an audible click. As such, the internal surface of the C-shaped connector corresponds to the outer surface of the ocular device 30 at the location of the interface features. In the embodiment shown, the C-shaped connector 100 includes two opposing flat surface sections 110 and 112 that correspond to the flat surfaces 84 and 86. See FIGS. 7 and 9. Connecting the flat surfaces 110 and 112 is a concave surface section 116 having a curvature that corresponds to the outer cylindrical surface of the ocular device. A lip or shoulder 120 on the inner surface of the connector springingly engages with the stepped portion 88 on the interface, with an audible click. See FIGS. 8 and 9. As shown in FIGS. 7 and 9, the C-shaped connector 100 may optionally include an orientation pin 124 positioned on the flat surface 110 and oriented so as to cooperatively interact with the groove 90 on the interface of the ocular. The holder 96 is preferably made of a material, such as Delrin® acetal resin from Dupont or other suitable polymer, and appropriately sized so that the lower flat surface 112 and shoulder 120 can spring open to accept the ocular device 30 and spring closed with an audible click when the stepped portion 88 of the ocular device 30 cooperates with the shoulder 120. The C-shaped connector 100 terminates at a release lever 128, which extends beyond the circumference of the ocular device 30 to allow a user to press it and decouple the ocular device 30 from the holder 96, as will be described in detail below.

Securely connecting the ocular device 30 to the holder 96 will now be described with reference to FIGS. 7-12. In use, the ocular device 30 can be inserted sideways into the holder 96 by simply aligning the groove 90 on the ocular device 30 with the pin 124 of the C-shaped connector 100 and then pushing the ocular device 30 into the opening 102 of the holder 96 until it locks into place with an audible click. It will be appreciated that the interaction between the stepped portion 88 of the interface on the ocular device 30 and the shoulder 120 of the connection section 100 locks the ocular device 30 in place and assists in preventing the ocular device 30 from rotating with respect to the holder 96.

To remove the ocular device 30 from the holder 96, the release lever 128 is pulled away from the ocular device 30 so that the opening 102 in the C-shaped connector 100 widens and the stepped portion 88 becomes disengaged from the shoulder 120. The ocular lens device 30 can then be pulled out of the C-shaped connector 100 of the holder 96.

As will be appreciated, the interface on the ocular device 30 and the C-shaped connector 100 include one or more cooperating parts that hold the connector to the ocular. The position of the cooperating parts could be reversed. For example, the C-shaped connector could include the stepped section 88 while the interface on the ocular could include an outwardly extending shoulder that fits within the stepped section 88. Furthermore the interface of the ocular C-shaped connector could include fewer or more than two flat sections.

One application of the probe/lens device and/or connector would be in concert with a single use steerable catheter, and mother scope during an ERCP procedure, such as described in U.S. patent application Ser. No. 11/089,520, filed Mar. 23, 2005, which is hereby incorporated by reference.

The principles, preferred embodiments, and modes of operation of the present invention have been described in the foregoing description. However, the invention which is intended to be protected is not to be construed as limited to the particular embodiments disclosed. Further, the embodiments described herein are to be regarded as illustrative rather than restrictive. Variations and changes may be made by others, and equivalents employed, without departing from the scope of the present invention. Accordingly, it is expressly intended that all such variations, changes, and equivalents fall within the scope of the present invention. For example, as described above and shown herein, the visualization probe was formed with the male portion of the connector and the ocular device was formed with the female portion of the connector. However, it will be appreciated that these roles may be reversed such that the visualization probe is formed with the female portion and the ocular device is formed with the male portion.

Furthermore, although the proximal connector includes both illumination and imaging fibers, it will be appreciated that the present invention is useful with a connector that has only imaging fibers, only illumination fibers, both illumination and imaging fibers, or other optical components.

The embodiments of the invention in which an exclusive property or privilege is claimed are defined as follows:

1. A connector for securing an ocular to a proximal connector on a visualization probe comprising:
a post positioned on one of the ocular or the proximal connector;
a bore positioned on the other of the proximal connector or ocular; and
a slot positioned either on the post or in the bore that includes a circumferentially extending path and a protrusion that fits within the slot such that when one or both of the post and bore are rotated with respect to each other, the post is secured within the bore.

2. The connector of claim 1, wherein the circumferentially extending path of the slot is positioned at the end of a section of the slot that extends lengthwise along the post or in the bore.

3. The connector of claim 1, wherein the circumferentially extending path is partially helical.

4. The connector of claim 1, wherein the post is releasably received within the bore.

5. The connector of claim 1, wherein the protrusion is a pin.

6. The connector of claim 1, wherein the post is on the proximal connector and the bore is in the ocular.

7. The connector of claim 1, wherein the post is on the ocular and the bore is in the proximal connector.

8. The connector of claim 6, wherein the slot is on the post and the pin is in the bore.

9. The connector of claim 6, wherein the slot is in the bore and the pin is on the post.

10. The connector of claim 7, wherein the slot is on the post and the pin is in the bore.

11. The connector of claim 7, wherein the slot is in the bore and the pin is on the post.

12. A holder for releasably engaging an ocular for use with a visualization probe, comprising:
a shaft; and
an elastic, C-shaped connector at the end of the shaft that has a generally arcuate inner surface that mates with a generally cylindrical surface of the ocular, wherein the C-shaped connector and the ocular include a cooperating recess and a shoulder that springingly engage so that the C-shaped connector is secured to the ocular.

13. The holder of claim 12, wherein the ocular includes the recess and the C-shaped connector includes the shoulder that springingly engages the recess.

14. The holder of claim 12, wherein the ocular includes an outwardly extending shoulder and the C-shaped connector includes an inwardly extending recess that springingly receives the shoulder of the ocular.

15. The holder of claim 12, wherein the holder includes one or more flat surfaces on the interior surface of the C-shaped connector that engage a corresponding one or more flat surfaces on the ocular.

16. The holder of claim 15, wherein one or more of the flat surfaces on the C-shaped connector and the ocular include a cooperating pin and groove oriented transverse to the longitudinal axis of the ocular to guide the C-shaped connector onto the ocular.

17. The holder of claim 12, wherein the C-shaped connector includes an outwardly extending tab positioned at the end of connector that is bendable to disengage the shoulder and recess when the connector is positioned on the ocular.

18. The holder of claim 12, wherein the recess and shoulder springingly engage so that the C-shaped connector is releasably secured to the ocular.

19. A visualization system, comprising:
an ocular;
an optical shaft having a proximal connector, wherein the connector includes:
a post positioned on one of the ocular or the proximal connector;
a bore positioned on the other of the proximal connector or ocular; and
a slot positioned either on the post or in the bore that includes a circumferentially extending path and a protrusion that fits within the slot such that when one or both of the post and bore are rotated with respect to each other, the post is secured within the bore.

20. The visualization system of claim 19, wherein the circumferentially extending path of the slot is positioned at the end of a section of the slot that extends lengthwise along the post or in the bore.

21. The visualization system of claim 19, wherein the circumferentially extending path is partially helical.

22. The visualization system of claim 19, wherein the post is releasably received within the bore.

23. The visualization system of claim 19, wherein the protrusion is a pin.

24. The visualization system of claim 19, wherein the post is on the proximal connector and the bore is in the ocular.

25. The visualization system of claim 19, wherein the post is on the ocular and the bore is in the proximal connector.

26. The visualization system of claim 24, wherein the slot is on the post and the pin is in the bore.

27. The visualization system of claim 24, wherein the slot is in the bore and the pin is on the post.

28. The visualization system of claim 25, wherein the slot is on the post and the pin is in the bore.

29. The visualization system of claim 25, wherein the slot is in the bore and the pin is on the post.
